(19) United States
(12) Patent Application Publication
(10) Pub. No.: US 2024/0347174 A1
(43) Pub. Date: Oct. 17, 2024

(54) MEDICAL IMAGE PROCESSING APPARATUS AND RECORDING MEDIUM

(71) Applicant: CANON MEDICAL SYSTEMS CORPORATION, Tochigi (JP)

(72) Inventors: Hiroshizu MORISHIMA, Utsunomiya (JP); Junya SUZUKI, Utsunomiya (JP); Taisuke IWAMURA, Utsunomiya (JP); Keita NAKAGOMI, Utsunomiya (JP)

(73) Assignee: CANON MEDICAL SYSTEMS CORPORATION, Tochigi (JP)

(21) Appl. No.: 18/630,023

(22) Filed: Apr. 9, 2024

(30) Foreign Application Priority Data
Apr. 12, 2023 (JP) 2023-065179
Apr. 3, 2024 (JP) 2024-060117

(51) Int. Cl. G16H 30/40

(52) U.S. Cl. CPC G16H 30/40

(57) ABSTRACT

A medical image processing apparatus according to the present embodiment includes processing circuitry. The processing circuitry acquires a first image and a second image with respect to a target site of a subject, the first image and the second image being medical images with different imaging times. The processing circuitry sets a plurality of regions of interest in the first image and the second image. The processing circuitry specifies pairs of corresponding regions of interest in the first image and the second image among the plurality of regions of interest. The processing circuitry causes a display to display the first image and the second image, and causes the display to display information corresponding to each of the plurality of regions of interest between the first image and the second image in a manner such that regions of interest for which the pair has been specified are distinguished from regions of interest for which the pair has not been specified.

### CROSS-REFERENCE TO RELATED APPLICATIONS

This application is based upon and claims the benefit of priority from Japanese Patent Application No. 2023-065179, filed on Apr. 12, 2023; and Japanese Patent Application No. 2024-060117, filed on Apr. 3, 2024, the entire contents of which are incorporated herein by reference.

### FIELD

Embodiments described herein relate generally to a medical image processing apparatus and a recording medium.

### BACKGROUND

A medical imaging diagnostic apparatus, for example, takes an image of a subject to generate a three-dimensional medical image including a plurality of slice images of the subject. A physician (for example, a radiologist), who is a user, performs reading by using the three-dimensional medical image. For example, a radiologist performs reading by using a plurality of medical images (slice images) and prepares a reading report. When performing the reading, the radiologist may perform comparative reading by comparing a medical image (previous image) obtained by imaging the subject in a previous examination with a medical image (current image) obtained by imaging the subject in a current examination. In doing so, the radiologist needs to compare the sizes of regions of interest (ROI) of the subject in the previous image and the current image. However, when the radiologist compares the sizes of the ROI in the previous image and the current image, much effort is required.

### BRIEF DESCRIPTION OF THE DRAWINGS

FIG. 1 is a diagram illustrating an example of a configuration of a medical image processing system including a medical image processing apparatus according to a first embodiment;

FIG. 2 is a diagram illustrating an example of a configuration of the medical image processing apparatus according to the first embodiment;

FIG. 3 is a flowchart illustrating the procedure of a process performed by the medical image processing apparatus according to the first embodiment;

FIG. 4 is a diagram illustrating a display example by the medical image processing apparatus according to the first embodiment;

FIG. 5 is a diagram for explaining an enlarged display process performed by the medical image processing apparatus according to the first embodiment;

FIG. 6 is a diagram illustrating a display example by a medical image processing apparatus according to a second embodiment;

FIG. 7 is a diagram illustrating an example of a configuration of a medical image processing apparatus according to a third embodiment; and

FIG. 8 is a diagram illustrating a display example by the medical image processing apparatus according to the third embodiment, and is a diagram for explaining a supplementary information display process.

### DETAILED DESCRIPTION

A medical image processing apparatus according to the present embodiment includes processing circuitry. The processing circuitry acquires a first image and a second image with respect to a target site of a subject, the first image and the second image being medical images with different imaging times. The processing circuitry sets a plurality of regions of interest in the first image and the second image. The processing circuitry specifies pairs of corresponding regions of interest in the first image and the second image among the plurality of regions of interest. The processing circuitry causes a display to display the first image and the second image, and causes the display to display information corresponding to each of the plurality of regions of interest between the first image and the second image in a manner such that regions of interest for which the pair has been specified are distinguished from regions of interest for which the pair has not been specified.

Embodiments of a medical image processing apparatus and a recording medium are described below in detail with reference to the drawings. The following is an example of a medical image processing system including the medical image processing apparatus.

### First Embodiment

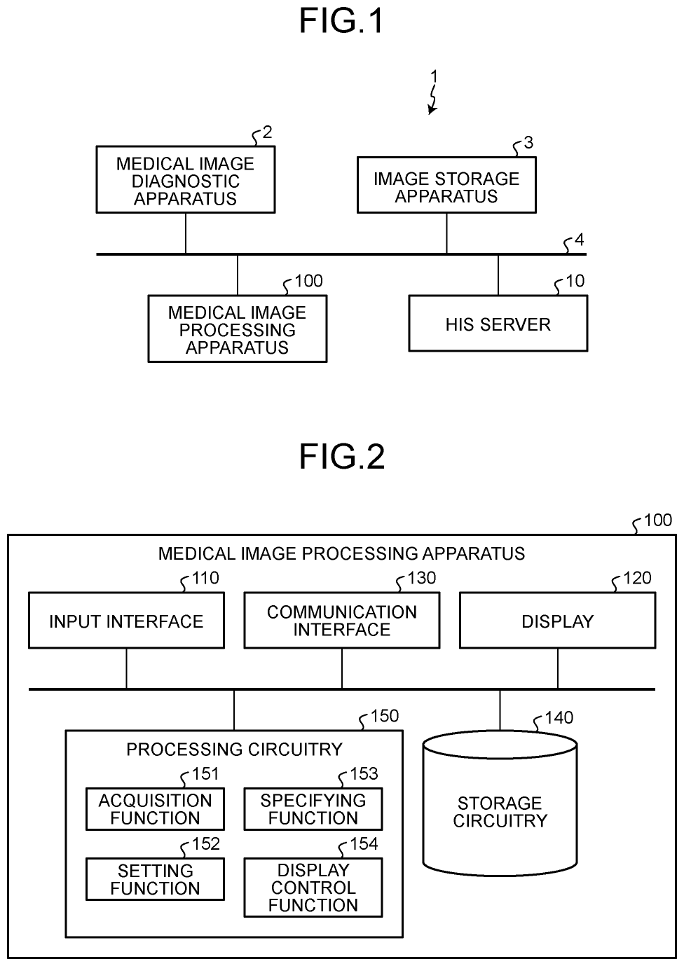

FIG. 1 is a diagram illustrating an example of a configuration of a medical image processing system 1 including a medical image processing apparatus 100 according to a first embodiment. The medical image processing system 1 illustrated in FIG. 1 includes the medical image processing apparatus 100, a medical image diagnostic apparatus 2, an image storage apparatus 3, and a hospital information system (HIS) server 10. The medical image processing apparatus 100 is connected to the medical image diagnostic apparatus 2, the image storage apparatus 3, and the HIS server 10 by a network 4 such as an in-hospital local area network (LAN) installed in a hospital, for example. The apparatuses are capable of communicating with one another directly or indirectly. For example, when a picture archiving and communication system (PACS) is introduced into the medical image processing system 1, the apparatuses transmit and receive a medical image and the like in accordance with a digital imaging and communications in medicine (DICOM) standard.

The HIS server 10 manages information generated in the hospital. The information generated in the hospital includes patient information, examination order information, and the like. The patient information includes patient's basic information and medical information, and information on examinations performed. The basic information includes patient ID, name, date of birth, gender, blood type, height, weight, and the like. Identification information for uniquely identifying a patient is set in the patient ID. The patient medical information includes information such as numerical values (measured values) and medical records, and information indicating the date and time of recording of the information. Examples of the patient medical information include drug prescriptions by physicians, nursing records by nurses, examinations performed by a laboratory department, and meal arrangements at the time of hospitalization. For example, the prescriptions are recorded in electronic medical records by the physicians, and the nursing records are recorded in the electronic medical records by the nurses. The information on examinations performed includes information on examinations performed in the past and the results of such examinations, and information indicating the date of execution of such examinations.

The medical image diagnostic apparatus 2 is an X-ray diagnostic apparatus, an X-ray computed tomography (CT) apparatus, a magnetic resonance imaging (MRI) apparatus, an ultrasonic diagnostic apparatus, or a single photon emission computed tomography (SPECT) apparatus. The medical image diagnostic apparatus 2 is a positron emission computed tomography (PET) apparatus, a SPECT-CT apparatus in which a SPECT apparatus and an X-ray CT apparatus are integrated, a PET-CT apparatus in which a PET apparatus and an X-ray CT apparatus are integrated, a group of these apparatuses, or the like. The medical image diagnostic apparatus 2 can generate a two-dimensional medical image, a three-dimensional medical image (volume data), a time-series two-dimensional medical image, and a time-series three-dimensional medical image.

On the basis of the patient information and the examination order information from the HIS server 10, the medical image diagnostic apparatus 2 collects medical images by capturing an image of a subject. For example, an X-ray CT apparatus serving as the medical image diagnostic apparatus 2 moves an X-ray tube and an X-ray detector in a swiveling motion around a subject to which a contrast agent has been administered, detects X-rays transmitted through the subject, and collects projection data. On the basis of the collected projection data, the X-ray CT apparatus generates a two-dimensional CT image, a three-dimensional CT image (volume data), a time-series two-dimensional CT image, or a time-series three-dimensional CT image. Alternatively, on the basis of the collected projection data, the X-ray CT apparatus generates a plurality of two-dimensional CT images along predetermined directions. For example, the X-ray CT apparatus generates a two-dimensional CT image of a plurality of axial cross sections following a body axial direction.

The medical image diagnostic apparatus 2 transmits the generated medical images to the image storage apparatus 3. When transmitting the medical images to the image storage apparatus 3, the medical image diagnostic apparatus 2 transmits supplementary information such as a patient ID for identifying a patient, an examination ID for identifying an examination, a device ID for identifying the medical image diagnostic apparatus 2, a series ID for identifying one-time imaging by the medical image diagnostic apparatus 2, and the like.

The image storage apparatus 3 is a database that stores the medical images. Specifically, the image storage apparatus 3 includes storage circuitry, and stores the medical images transmitted from the medical image diagnostic apparatus 2 in the storage circuitry, thereby storing the medical images. The storage circuitry of the image storage apparatus 3 is, for example, a semiconductor memory element such as a random access memory (RAM) and a flash memory, or a storage device such as a hard disk and an optical disk. The medical images stored in the image storage apparatus 3 are stored in association with the patient ID, the examination ID, the device ID, the series ID, and the like. Therefore, the medical image processing apparatus 100 can acquire necessary medical images from the image storage apparatus 3 by performing a search using the patient ID, the examination ID, the device ID, the series ID, and the like.

The medical image processing apparatus 100 is an image processing apparatus that performs image processing on the medical images, such as a workstation, a picture archiving and communication system (PACS) image server or viewer, various apparatuses in an electronic medical record system, and the like. The medical image processing apparatus 100 performs various processes on the medical images acquired from the medical image diagnostic apparatus 2 or the image storage apparatus 3. In the present embodiment, the medical image processing apparatus 100 is an apparatus used by a physician (for example, a radiologist) to observe or read medical images.

FIG. 2 is a diagram illustrating an example of the configuration of the medical image processing apparatus 100 according to the first embodiment. As illustrated in FIG. 2, the medical image processing apparatus 100 includes an input interface 110, a display 120, a communication interface 130, storage circuitry 140, and processing circuitry 150.

The input interface 110 has a pointing device such as a mouse, keyboard, and the like, receives input of various operations for the medical image processing apparatus 100 from a physician who is a user, and transmits information on instructions and settings received from the user to the processing circuitry 150.

The display 120 is a monitor referred to by the user. Under the control of the processing circuitry 150, the display 120 displays images to the user or displays a graphical user interface (GUI) for receiving various instructions, various settings, and the like from the user via the input interface 110. The communication interface 130 is a network interface card (NIC) or the like, and communicates with other devices. The display 120 is an example of a display.

The storage circuitry 140 is, for example, a semiconductor memory element such as a RAM or a flash memory, or a storage device such as a hard disk or an optical disk. The storage circuitry 140 stores three-dimensional medical images being the medical images acquired from the medical image diagnostic apparatus 2 or the image storage apparatus 3 and including a plurality of slice images of a subject.

The processing circuitry 150 controls the components of the medical image processing apparatus 100. For example, as illustrated in FIG. 2, the processing circuitry 150 performs an acquisition function 151, a setting function 152, a specifying function 153, and a display control function 154. For example, respective processing functions performed by the acquisition function 151, the setting function 152, the specifying function 153, and the display control function 154, which are the components of the processing circuitry 150, are stored in the storage circuitry 140 in the form of computer programs executable by a computer. The processing circuitry 150 is a processor that reads the computer programs from the storage circuitry 140 and executes the read computer programs, thereby implementing functions corresponding to the executed computer programs. In other words, the processing circuitry 150 in the state of having read the computer programs has the functions illustrated in the processing circuitry 150 in FIG. 2. The acquisition function 151, the setting function 152, the specifying function 153, and the display control function 154 are examples of an “acquisition unit”, a “setting unit”, a “specifying unit”, and a “display control unit”, respectively.

The term “processor” used in the above description means, for example, circuitry such as a central processing unit (CPU), a graphics processing unit (GPU), or an application specific integrated circuit (ASIC). The term “processor” means circuitry such as a programmable logic device. Examples of the programmable logic device include a simple programmable logic device (SPLD) and a complex programmable logic device (CPLD). Examples of the programmable logic device include a field programmable gate array (FPGA). When the processor is, for example, a CPU, the processor reads out and executes the computer programs stored in the storage circuitry 140 to implement the functions. On the other hand, when the processor is, for example, an ASIC, the computer programs are directly incorporated in the circuitry of the processor instead of storing the computer programs in the storage circuitry 140. Each processor of the present embodiment is not limited to being configured as a single piece of circuitry for each processor, and one processor may be configured by combining a plurality of pieces of independent circuitry to implement the functions thereof. The plurality of components in FIG. 2 may be integrated into one processor to implement the functions thereof.

As described above, the storage circuitry 140 stores the three-dimensional medical images being the medical images acquired from the medical image diagnostic apparatus 2 or the image storage apparatus 3 and including the plurality of slice images of the subject. A physician (for example, a radiologist), who is a user, performs reading by using the three-dimensional medical image (the plurality of slice images), and prepares a reading report. When performing the reading, the user may perform comparative reading by comparing a medical image (previous image) obtained by imaging the subject in a previous examination with a medical image (current image) obtained by imaging the subject in a current examination. In doing so, the user needs to compare the sizes of regions of interest (ROI) of the subject in the previous image and the current image. However, when the user compares the sizes of the ROI in the previous image and the current image, much effort is required. As a result, the efficiency of reading may not be improved.

Therefore, the medical image processing apparatus 100 according to the present embodiment performs the following processes in order to improve the efficiency of reading. First, in the medical image processing apparatus 100 according to the present embodiment, the acquisition function 151 acquires a first image and a second image, which are medical images with different imaging times, with respect to a target site of a subject. The setting function 152 sets a plurality of regions of interest in the first image and the second image. The specifying function 153 specifies pairs of corresponding regions of interest in the first image and the second image among the plurality of regions of interest. The display control function 154 causes the display 120 to display the first image and the second image, and causes the display 120 to display information corresponding to each of the plurality of regions of interest between the first image and the second image in a manner that distinguishes regions of interest for which the pair has been specified from regions of interest for which the pair has not been specified.

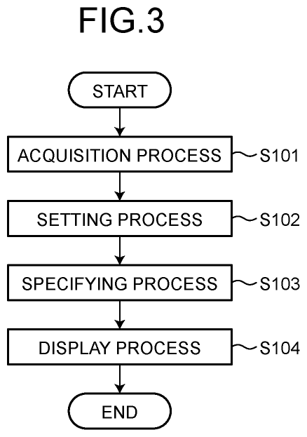

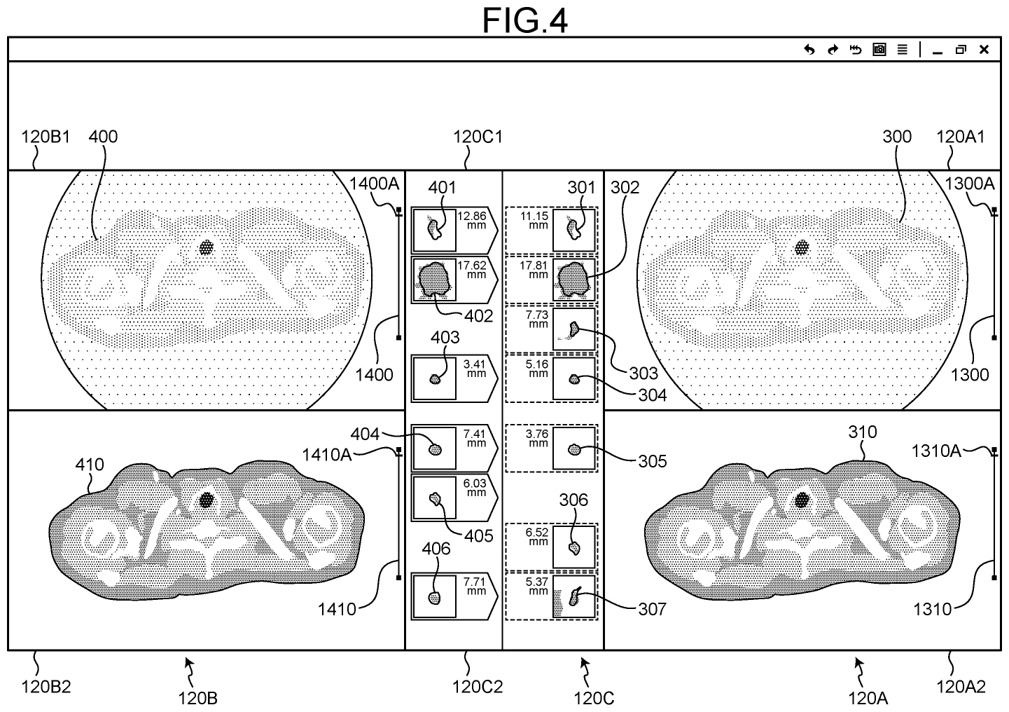

Processes performed by the medical image processing apparatus 100 according to the first embodiment are described below with reference to FIGS. 3 and 4. FIG. 3 is a flowchart illustrating the procedure of a process performed by the medical image processing apparatus 100 according to the present embodiment. FIG. 4 is a diagram illustrating a display example of the medical image processing apparatus 100 according to the first embodiment.

Step S101 in FIG. 3 is a step performed by the processing circuitry 150 that calls a computer program corresponding to the acquisition function 151 from the storage circuitry 140 and executes the read computer program. At step S101, the acquisition function 151 acquires a current image and a previous image, which are medical images with different imaging times, with respect to a target site of the subject. For example, the acquisition function 151 acquires, from the image storage apparatus 3, along with the current image and the previous image, patient information including a patient ID, a patient name, and the like for identifying the subject (patient), the date the current image and the previous image have been taken, and the like.

The current image is a medical image obtained by imaging the subject in a current examination, and the previous image is a medical image obtained by imaging the subject in a previous examination. The current image is an example of the “first image” and the previous image is an example of the “second image”.

For example, suppose that the medical image is a three-dimensional CT image (volume data) including a plurality of medical image data (reconstructed images including a plurality of axial cross-sections) obtained by helical scanning the subject with an X-ray CT apparatus being the medical image diagnostic apparatus 2. For example, the current image and the previous image are medical images of the subject taken by the X-ray CT apparatus at the time of examination, under scanning conditions such as an X-ray tube voltage (tube voltage) of 120 kV and a slice thickness of 7 mm.

For example, suppose that the target site of the subject is the lungs. In this case, for example, the acquisition function 151 acquires an image of a lung field condition and an image of a mediastinal condition as the current image, and acquires an image of the lung field condition and an image of the mediastinal condition as the previous image. The image of the lung field condition and the image of the mediastinal condition are generated from the same projection data. The image of the lung field condition and the image of the mediastinal condition may be generated by using a modality (for example, the medical image diagnostic apparatus 2) such as an X-ray CT apparatus or by using a workstation (for example, the medical image processing apparatus 100). When the images are generated by the workstation, the projection data may be reconstructed by the workstation to generate an image of any location. Alternatively, a three-dimensional CT image (volume data) of the lung field may be generated on the modality side, and a multi planer reconstruction (MPR) image may be cut from the volume data by the workstation to generate an image of any location.

The image of the lung field condition is an image in which a window width and a window level are set in order to observe the lung field (lungs and surroundings of the lungs) including air, in the CT image. The image of the lung field condition, which is stripped of bone and soft tissue, is suitable for observing minute changes in the lungs.

The image of the mediastinal condition is an image in which a window width and a window level are focused on soft tissues constituting the mediastinum, in the CT image. The image of the mediastinal condition is suitable for observing the heart, esophagus, and the like although the structure of the lungs is blacked out.

Step S102 in FIG. 3 is a step performed by the processing circuitry 150 that reads a computer program corresponding to the setting function 152 from the storage circuitry 140 and executes the read computer program. At step S102, the setting function 152 sets a plurality of regions of interest in the current image and the previous image. For example, the region of interest is a nodule and corresponds to the ROI described above.

Regarding the setting of the region of interest by the setting function 152, the region of interest may be set automatically by a computer aided diagnosis (CAD) or manually by a user. The region of interest may also be set automatically and manually. An example in which the region of interest is manually set by the user is described in a first variation to be described below.

For example, the setting function 152 sets regions of interest 301 to 307 in the image of the lung field condition that is the current image (see FIG. 4). The setting function 152 further sets regions of interest 401 to 406 in the image of the lung field condition that is the previous image (see FIG. 4). At this time, the setting function 152 further automatically measures the diameters of the set regions of interest 301 to 307 and 401 to 406. For example, the setting function 152 automatically measures the diameters of the regions of interest 301 to 307 and 401 to 406 by the CAD.

For example, the setting function 152 sets the nodule as the region of interest in the image of the lung field condition. The image of the mediastinal condition is used as a reference image for determining whether an area set as the nodule in the image of the lung field condition is a nodule.

Step S103 in FIG. 3 is a step performed by the processing circuitry 150 that reads a computer program corresponding to the specifying function 153 from the storage circuitry 140 and executes the read computer program. At step S103, the specifying function 153 specifies pairs of corresponding regions of interest in the current image and the previous image among the plurality of regions of interest 301 to 307 and 401 to 406. That is, the specifying function 153 ties the region of interest in the previous image to the region of interest in the current image.

The specifying function 153, for example, specifies pairs of corresponding regions of interest in the current image and the previous image by using anatomical location information on the images.

For example, by using the anatomical location information of the images of the lung field condition being the current image and the previous image, the specifying function 153 specifies the region of interest 301 in the current image and the region of interest 401 in the previous image as a pair and specifies the region of interest 302 in the current image and the region of interest 402 in the previous image as a pair. For example, by using the anatomical location information of the images of the lung field condition being the current image and the previous image, the specifying function 153 specifies the region of interest 304 in the current image and the region of interest 403 in the previous image as a pair.

For example, by using the anatomical location information of the images of the lung field condition being the current image and the previous image, the specifying function 153 specifies a pair of the region of interest 305 in the current image and the region of interest 404 in the previous image and specifies a pair of the region of interest 307 in the current image and the region of interest 406 in the previous image.

The region of interest 303 in the image of the lung field condition, which is the current image, is a region of interest for which no pair has been specified. For example, the region of interest 303 is a newly set region of interest in the current image. The region of interest 306 in the image of the lung field condition, which is the current image, and the region of interest 405 in the image of the lung field condition, which is the previous image, are regions of interest for which no pair has been specified. For example, the region of interest 306 is a newly set region of interest in the current image, and the region of interest 405 in the previous image is an area having no corresponding area in the current image.

Step S104 in FIG. 3 is a performed by the processing circuitry 150 that reads a computer program corresponding to the display control function 154 from the storage circuitry 140 and executes the read computer programs. At step S104, the display control function 154 causes the display 120 to display the current image and the previous image, and also causes the display 120 to display information corresponding to each of the plurality of regions of interest between the current image and the previous image in a manner that distinguishes the regions of interest for which the above pair has been specified from the regions of interest for which the above pair has not been specified.

Specifically, as illustrated in FIG. 4, the display control function 154 causes the display 120 to display display areas 120A, 120B, and 120C. The display area 120C is a display area located between the display area 120A and the display area 120B. For example, the display areas 120A and 120B are located on the right and left sides of the display 120, respectively, and the display area 120C is located in the center portion of the display 120.

In the example illustrated in FIG. 4, the display control function 154 displays an image 300 of the lung field condition and an image 310 of the mediastinal condition, which are the current images, in the display area 120A.

Specifically, the display control function 154 displays the image 300 of the lung field condition and the image 310 of the mediastinal condition in an upper stage 120A1 and a lower stage 120A2 of the display area 120A, respectively. In the upper stage 120A1 and the lower stage 120A2 of the display area 120A, the display control function 154 displays, along with the current images, patient information including a patient ID, a patient name, and the like for identifying the subject (patient), the date the current images have been taken, and parameters of scanning conditions such as an axial section, a slice thickness of 7 mm, and a tube voltage of 120 kV. In the upper stage 120A1 of the display area 120A, the display control function 154 displays parameters of the lung field condition such as a window width and a window level, along with the image 300 of the lung field condition being the current image. In the lower stage 120A2 of the display area 120A, the display control function 154 displays parameters of the mediastinum condition such as a window width and a window level, along with the image 310 of the mediastinum condition being the current image.

In the upper stage 120A1 of the display area 120A, the display control function 154 displays a scroll bar 1300 and a knob 1300A located on the scroll bar 1300, along with the image 300 of the lung field condition. In the lower stage 120A2 of the display area 120A, the display control function 154 displays a scroll bar 1310 and a knob 1310A located on the scroll bar 1310, along with the image 310 of the mediastinal condition. The knobs 1300A and 1310A are bars serving as what is called “knobs”, and indicate locations where the image 300 of the lung field condition and the image 310 of the mediastinal condition are currently displayed, respectively.

In the example illustrated in FIG. 4, the display control function 154 displays an image 400 of the lung field condition and an image 410 of the mediastinal condition, which are the previous images, in the display area 120B.

Specifically, the display control function 154 displays the image 400 of the lung field condition and the image 410 of the mediastinal condition in an upper stage 120B1 and a lower stage 120B2 of the display area 120B, respectively. In the upper stage 120B1 and the lower stage 120B2 of the display area 120B, the display control function 154 displays, along with the previous images, the patient information including a patient ID, a patient name, and the like for identifying the subject (patient), the date the previous images have been taken, and the parameters of scanning conditions such as an axial section, a slice thickness of 7 mm, and a tube voltage of 120 kV. In the upper stage 120B1 of the display area 120B, the display control function 154 displays parameters of the lung field condition such as a window width and a window level, along with the image 400 of the lung field condition being the previous image. In the lower stage 120B2 of the display area 120B, the display control function 154 displays parameters of the mediastinum condition such as a window width and a window level, along with the image 410 of the mediastinum condition being the previous image.

In the upper stage 120B1 of the display area 120B, the display control function 154 displays a scroll bar 1400 and a knob 1400A located on the scroll bar 1400, along with the image 400 of the lung field condition. In the lower stage 120B2 of the display area 120B, the display control function 154 displays a scroll bar 1410 and a knob 1410A located on the scroll bar 1410, along with the image 410 of the mediastinal condition. The knobs 1400A and 1410A indicate locations where the image 300 of the lung field condition and the image 310 of the mediastinal condition are currently displayed, respectively.

A slice image at the same location is specified in the current image and the previous image. For example, the position of the slice image is managed by number. In the example illustrated in FIG. 4, the display control function 154 displays first slice images in the upper stage 120A1 and the lower stage 120A2 of the display area 120A as the image 300 of the lung field condition and the image 310 of the mediastinal condition, respectively. Similarly, in the example illustrated in FIG. 4, the display control function 154 displays first slice images in the upper stage 120B1 and the lower stage 120B2 of the display area 120B as the image 400 of the lung field condition and the image 410 of the mediastinal condition, respectively.

Furthermore, suppose that a user operates the input interface 110 to move a knob in any one of the four scroll bars 1300, 1310, 1400, and 1410. In this case, the display control function 154 displays a slice image at a position corresponding to the amount of movement of the operated knob, and also links the knobs of the remaining scroll bars to display a slice image at a position corresponding to the same amount of movement as the amount of movement of the operated knob.

For example, when the user operates the input interface 110 to move the knob 1300A in the upper stage 120A1 of the display area 120A, the display control function 154 displays a slice image at a position corresponding to the amount of movement of the knob 1300A in the upper stage 120A1 of the display area 120A. At this time, the display control function 154 also links the knob 1310A of the scroll bar 1310 to display, in the lower stage 120A2 of the display area 120A, a slice image at a position corresponding to the same amount of movement as the amount of movement of the operated knob 1300A. Similarly, the display control function 154 also links the knob 1400A of the scroll bar 1400 to display, in the upper stage 120B1 of the display area 120B, a slice image at a position corresponding to the same amount of movement as the amount of movement of the operated knob 1300A. Similarly, the display control function 154 also links the knob 1410A of the scroll bar 1410 to display, in the lower stage 120B2 of the display area 120B, a slice image at a position corresponding to the same amount of movement as the amount of movement of the operated knob 1300A.

In any one of the four scroll bars 1300, 1310, 1400, 1410, the display control function 154 also links the knobs of the remaining scroll bars according to the amount of movement of the operated knob; however, the user can, by an instruction on the input interface 110, release the linking process described above. For example, when the user operates the input interface 110 to move the knobs 1300A, 1310A, 1400A, and 1410A, respectively, the display control function 154 displays the slice images at positions corresponding to the amounts of movement of the operated knobs.

In the example illustrated in FIG. 4, the display control function 154 causes the display area 120C (center of screen) of the display 120 to display, as thumbnail images, images obtained by cutting out the plurality of regions of interest 301 to 307 and 401 to 406 set by the setting function 152 from the images of the lung field condition. The display control function 154 may further cause the display area 120C to display, as a thumbnail image, an image obtained by cutting out the same region as the region of interest from the image of the mediastinal condition, along with the above thumbnail images. The display control function 154 may also cause the display area 120C to display, as a thumbnail image, only an image obtained by cutting out the same region as the region of interest from the image of the mediastinum condition.

For example, in FIG. 4, the display control function 154 displays information corresponding to each of the plurality of regions of interest 301 to 307 and 401 to 406 in the display area 120C in a manner that the regions of interest 301 and 401, the regions of interest 302 and 402, the regions of interest 304 and 403, the regions of interest 305 and 404, and the regions of interest 307 and 406, for which the above pair has been specified, are distinguished from the regions of interest 303, 306, and 405 for which the above pair has not been specified. In the example illustrated in FIG. 4, the display control function 154 displays the information corresponding to each of the plurality of regions of interest 301 to 307 and 401 to 406 so as to match the arrangement of the slice images in the current image and the previous image. Specifically, the display control function 154 arranges and displays the above information in a head-to-tail direction. Arranging the information in the head-to-tail direction is merely an example, and the display control function 154 may arrange and display the above information in a direction opposite to the head-to-tail direction or may display the above information in any order according to user's instructions by manual operation. The above information includes, for example, at least the diameter of the region of interest (nodule) and an image of the region of interest. The image of the region of interest is an image obtained by cutting out a corresponding region from the image of the lung field condition as described above; however, the image of the region of interest is not limited thereto and may be represented by an illustration of the corresponding region.

Specifically, in FIG. 4, the display control function 154 arranges and displays, in the display area 120C, pairs of information (diameters and images of nodules) corresponding to the regions of interest 301 and 401, the regions of interest 302 and 402, the regions of interest 304 and 403, the regions of interest 305 and 404, and the regions of interest 307 and 406 for which the above pair has been specified.

For example, the display control function 154 arranges and displays, in an upper stage 120C1 of the display area 120C, the pairs of the information (diameters and images of nodules) corresponding to the region of interest 301 in the current image and the region of interest 401 in the previous image as the region of interest for which the above pair has been specified in an I^{th} slice image (I is an integer of 2 or more) being the image of the lung field condition. In this case, the information corresponding to the region of interest 301 in the current image is displayed surrounded by a square line, and the information corresponding to the region of interest 401 in the previous image is displayed surrounded by a line in the shape of a home-based pentagon pointing to the right so as to point to the information corresponding to the region of interest 301 in the current image. Thus, in the image of the lung field condition, the user can easily compare the region of interest 301 in the current image with the region of interest 401 in the previous image, and further confirm that the diameter (11.15 mm) of the region of interest 301 in the current image has decreased compared to the diameter (12.86 mm) of the region of interest 401 in the previous image.

The display control function 154 further arranges and displays, in the upper stage 120C1 of the display area 120C, the pairs of the information (diameters and images of nodules) corresponding to the region of interest 302 in the current image and the region of interest 402 in the previous image as the region of interest for which the above pair has been specified in a J^{th} slice image (J is an integer satisfying I<J) being the image of the lung field condition. In this case, the information corresponding to the region of interest 302 in the current image is displayed surrounded by a square line, and the information corresponding to the region of interest 402 in the previous image is displayed surrounded by a pentagonal line so as to point to the information corresponding to the region of interest 302 in the current image. Thus, in the image of the lung field condition, the user can easily compare the region of interest 302 in the current image with the region of interest 402 in the previous image, and further confirm that the difference between the diameter (17.81 mm) of the region of interest 302 in the current image and the diameter (17.62 mm) of the region of interest 402 in the previous image is small.

The display control function 154 further arranges and displays, in the upper stage 120C1 of the display area 120C, the pairs of the information (diameters and images of nodules) corresponding to the region of interest 304 in the current image and the region of interest 403 in the previous image as the region of interest for which the above pair has been specified in an M^{th} slice image (M is an integer satisfying J<M) being the image of the lung field condition. In this case, the information corresponding to the region of interest 304 in the current image is displayed surrounded by a square line, and the information corresponding to the region of interest 403 in the previous image is displayed surrounded by a pentagonal line so as to point to the information corresponding to the region of interest 304 in the current image. Thus, in the image of the lung field condition, the user can easily compare the region of interest 304 in the current image with the region of interest 403 in the previous image, and further confirm that the diameter (5.16 mm) of the region of interest 304 in the current image has increased compared to the diameter (3.41 mm) of the region of interest 403 in the previous image.

The display control function 154 displays the region of interest 303 in the current image in the upper stage 120C1 of the display area 120C as the region of interest for which the above pair has not been specified in a K^{th} slice image (K is an integer satisfying J<K<M) being the image of the lung field condition. In this case, the information corresponding to the region of interest 303 in the current image is displayed surrounded by a square line, but there is no information pointed to by an arrow for that information. Thus, in the image of the lung field condition, the user can newly confirm the region of interest 303 in the current image, and confirm the diameter (7.73 mm) of the region of interest 303.

The display control function 154 further arranges and displays, in a lower stage 120C2 of the display area 120C, the pairs of the information (diameters and images of nodules) corresponding to the region of interest 305 in the current image and the region of interest 404 in the previous image as the region of interest for which the above pair has been specified in an N^{th} slice image (N is an integer satisfying M<N) being image of the lung field In this case, the information corresponding to condition. the region of interest 305 in the current image is displayed surrounded by a square line, and the information corresponding to the region of interest 404 in the previous image is displayed surrounded by a pentagonal line so as to point to the information corresponding to the region of interest 305 in the current image. Thus, in the image of the lung field condition, the user can easily compare the region of interest 305 in the current image with the region of interest 404 in the previous image, and further confirm that the diameter (3.76 mm) of the region of interest 305 in the current image has decreased compared to the diameter (7.41 mm) of the region of interest 404 in the previous image.

The display control function 154 further arranges and displays, in the lower stage 120C2 of the display area 120C, the pairs of the information (diameters and images of nodules) corresponding to the region of interest 307 in the current image and the region of interest 406 in the previous image as the region of interest for which the above pair has been specified in an R^{th} slice image (R is an integer satisfying N<R) being the image of the lung field condition. In this case, the information corresponding to the region of interest 307 in the current image is displayed surrounded by a square line, and the information corresponding to the region of interest 406 in the previous image is displayed surrounded by a pentagonal line so as to point to the information corresponding to the region of interest 307 in the current image. Thus, in the image of the lung field condition, the user can easily compare the region of interest 307 in the current image with the region of interest 406 in the previous image, and further confirm that the diameter (5.37 mm) of the region of interest 307 in the current image has decreased compared to the diameter (7.71 mm) of the region of interest 406 in the previous image.

The display control function 154 displays the region of interest 405 in the previous image in the lower stage 120C2 of the display area 120C as the region of interest for which the above pair has not been specified in a P^{th} slice image (P is an integer satisfying N<P) being the image of the lung field condition. In this case, the information corresponding to the region of interest 405 in the previous image is displayed surrounded by a line in the shape of a home-based pentagon pointing to the right, but the region of interest 405 in the previous image has no corresponding area in the current image. Thus, in the image of the lung field condition, the user can confirm that the region of interest 405 in the previous image has disappeared in a current examination.

The display control function 154 further displays the region of interest 306 in the current image in the lower stage 120C2 of the display area 120C as the region of interest for which the above pair has not been specified in a Q^{th} slice image (Q is an integer satisfying P<Q<R) being the image of the lung field condition. In this case, the information corresponding to the region of interest 306 in the current image is displayed surrounded by a square line, but there is no information pointed to by an arrow for that information. Thus, in the image of the lung field condition, the user can newly confirm the region of interest 306 in the current image, and confirm the diameter (6.52 mm) of the region of interest 306.

The user can further determine whether an area (region of interest) set as a nodule in the image of the lung field condition is a nodule by referring to the image of the mediastinal condition being a slice image at the same position as the image of the lung field condition.

In this way, in the medical image processing apparatus 100 according to the present embodiment, the acquisition function 151 acquires the current image (image of the lung field condition and image of the mediastinal condition) and the previous image (image of the lung field condition and image of the mediastinal condition), which are medical images with different imaging times, with respect to the target site (lung) of the subject. The setting function 152 sets a plurality of regions of interest in the current image and the previous image, and the specifying function 153 specifies pairs of corresponding regions of interest in the current image and the previous image. The display control function 154 causes the display 120 to display the current image and the previous image, and also causes the display 120 to display information (diameters and images of nodules) corresponding to each of the plurality of regions of interest between the current image and the previous image in a manner that distinguishes the regions of interest for which a pair has been specified from the regions of interest for which no pair has been specified. Thus, the display 120 displays the information (diameters and images of nodules) corresponding to each of the plurality of regions of interest, so that the user can easily compare the sizes of the regions of interest in the current image and the previous image when performing comparative reading. Therefore, in accordance with the medical image processing apparatus 100 according to the present embodiment, the efficiency of reading can be improved.

The medical image processing apparatus 100 according to the present embodiment displays the current image and the previous image in the display areas 120A and 120B of the display 120, respectively, and also displays information (diameters and images of nodules) corresponding to each of the plurality of regions of interest in the display area 120C between the display area 120A and the display area 120B of the display 120. In this way, in accordance with the medical image processing apparatus 100 according to the present embodiment, the information is displayed in the center portion of the display 120, thereby making it easier for the user visually recognize the information than when the information is displayed at a right end or a left end of the display 120.

### First Variation

The setting of the region of interest by the setting function 152 may be performed when a user (operator) designates an area in the current image (image of the lung field condition and image of the mediastinal condition) and the previous image (image of the lung field condition and image of the mediastinal condition) displayed on the display 120. In this case, the display control function 154 causes the display 120 to display an enlarged image corresponding to the area in response to the designation of area.

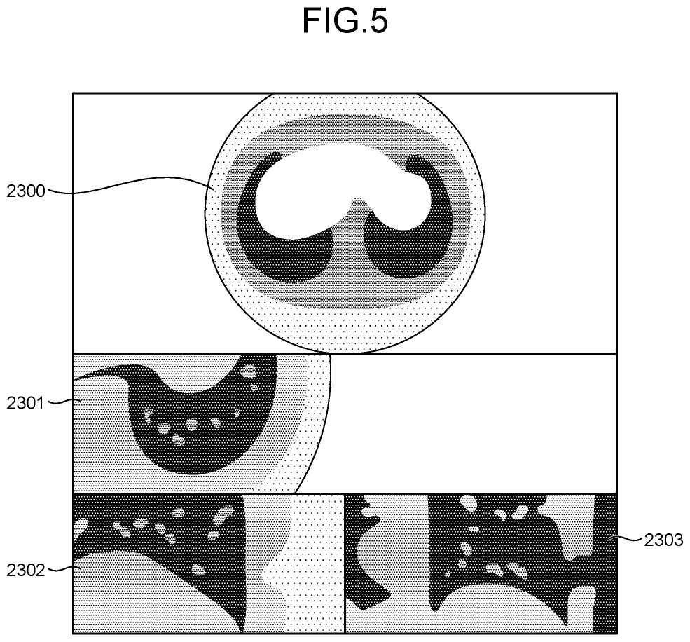

FIG. 5 is a diagram for explaining an enlarged display process performed by the medical image processing apparatus 100 according to the first embodiment.

For example, suppose that at step S104 in FIG. 3, when the display control function 154 is displaying the current image and the previous image on the display 120, the user performs a drag operation using the knobs 1300A and 1400A on the input interface 110, respectively. In this case, the display control function 154 displays slice images at positions corresponding to the amounts of movement of the drag operation in the upper stage 120A1 of the display area 120A and the upper stage 120B1 of the display area 120B, respectively. For example, the display control function 154 displays an L^{th} slice image (L is an integer satisfying K<L<M), which is the current image (image of the lung field condition) and the previous image (image of the lung field condition), in the upper stage 120A1 of the display area 120A and the upper stage 120B1 of the display area 120B, respectively, according to the amounts of movement of the drag operation.

For example, the user operates the input interface 110 to designate an area in the current image (image of the lung field condition) displayed in the upper stage 120A1 of the display area 120A. In this case, as illustrated in FIG. 5, the display control function 154 causes the display 120 to display a pop-up screen illustrated in FIG. 5 in response to the designation of area. The pop-up screen displays an axial section 2300 of the current image as the image of the lung field condition, and displays, as images in which designated areas are enlarged, an axial section 2301 with an enlarged designated area, a coronal section 2302 with an enlarged designated area, and a sagittal section 2303 with an enlarged designated area. For example, the user operates the input interface 110 to designate an area that is thought to be a nodule by referring to each of the sections 2301 to 2303 enlarged on the pop-up screen, and the setting function 152 newly sets the designated area as a region of interest.

Similarly, the user operates the input interface 110 to designate an area in the previous image (image of the lung field condition) displayed in the upper stage 120B1 of the display area 120B, and the setting function 152 newly sets the designated area as a region of interest.

Regarding the setting of the region of interest by the setting function 152, the region of interest may be automatically set by the CAD, or may be manually set by the user as described above. The region of interest may also be set automatically and manually.

In this way, in the medical image processing apparatus 100 according to the present embodiment, when the user designates areas in the current image (image of the lung field condition and image of the mediastinal condition) and the previous image (image of the lung field condition and image of the mediastinal condition), the setting function 152 newly sets the designated areas as regions of interest. Subsequently, the specifying function 153 specifies pairs of the newly set regions of interest, and the display control function 154 arranges and displays, in the upper stage 120C1 of the display area 120C, the pairs of the newly specified regions of interest. Thus, even for an area designated by the user, information (diameters and images of nodules) corresponding to the region of interest for which a pair has been newly specified is displayed on the display 120, so that when performing comparative reading, the user can easily compare the sizes of regions of interest in the previous image and the current image. Therefore, in accordance with the medical image processing apparatus 100 according to the present embodiment, the efficiency of reading can be improved.

### Second Variation

A reception display process performed by the medical image processing apparatus 100 according to the first embodiment is described below.

First, the input interface 110 receives the selection of information (diameters and images of nodules) made by the user who is an operator. The input interface 110 is an example of a “reception unit”.

For example, at least one of the current image (image of the lung field condition and image of the mediastinal condition) and the previous image (image of the lung field condition and image of the mediastinal condition) is a three-dimensional medical image obtained by imaging a three-dimensional area of the subject. Therefore, when a region of interest corresponding to the information (diameters and images of nodules) for which the selection has been received by the input interface 110 is a region set by the setting function 152 in the three-dimensional medical image, the display control function 154 causes the display 120 to display a cross-section including the region of interest in the three-dimensional medical image.

For example, suppose that at step S104 in FIG. 3, when the display control function 154 is displaying the current image and the previous image on the display 120, the user selects information (diameters and images of nodules) of the region of interest 301 in the current image by using the input interface 110. In this case, the input interface 110 receives the selection of the information (diameters and images of nodules) of the region of interest 301 made by the user. At this time, the display control function 154 displays an image of the lung field condition being an I^{th} slice image in the upper stage 120A1 of the display area 120A as a cross-section (current image) including the region of interest 301.

When the display control function 154 displays the I^{th} slice image as the cross-section including the region of interest in any one display area of the upper stage 120A1 and the lower stage 120A2 of the display area 120A and the upper stage 120B1 and the lower stage 120B2 of the display area 120B, the display control function 154 also displays the I^{th} slice image also in the remaining display areas.

For example, when the input interface 110 receives the selection of the information (diameters and images of nodules) of the region of interest 301 made by the user, the display control function 154 displays an image of the lung field condition, which is the I^{th} slice image, as the cross-section (current image) including the region of interest 301 in the upper stage 120A1 of the display area 120A. At this time, the display control function 154 displays an image of the mediastinal condition, which is the I^{th} slice image, as the current image also in the lower stage 120A2 of the display area 120A. Similarly, the display control function 154 displays the image of the lung field condition, which is the I^{th} slice image, as the previous image also in the upper stage 120B1 of the display area 120B. Similarly, the display control function 154 displays the image of the mediastinal condition, which is the I^{th} slice image, as the previous image also in the lower stage 120B2 of the display area 120B.

As described above, the medical image processing apparatus 100 according to the present embodiment causes the display 120 to display a cross-section including a region of interest corresponding to the information (diameters and images of nodules) for which the selection has been received, so that the user can easily confirm the cross-section including the region of interest in the previous image and the current image when performing comparative reading. Therefore, in accordance with the medical image processing apparatus 100 according to the present embodiment, the efficiency of reading can be improved.

### Second Embodiment

In the second embodiment, the same explanations as in the first embodiment are omitted. The medical image processing apparatus 100 according to the second embodiment causes the display 120 to display a medical image (current image) obtained by imaging a subject in a current examination and a medical image (image n times before) obtained by imaging the subject in an examination n times before (n is an integer of 1 or more) so that the images can be compared and read.

For example, when N is 2, the medical image processing apparatus 100 according to the second embodiment causes the display 120 to display the current image and a medical image (image two times before) obtained by imaging the subject in an examination two times before so that the images can be compared and read. For example, when N is 1, the medical image processing apparatus 100 according to the second embodiment causes the display 120 to display the current image and a previous image (image one time before) so that the images can be compared and read, as in the first embodiment.

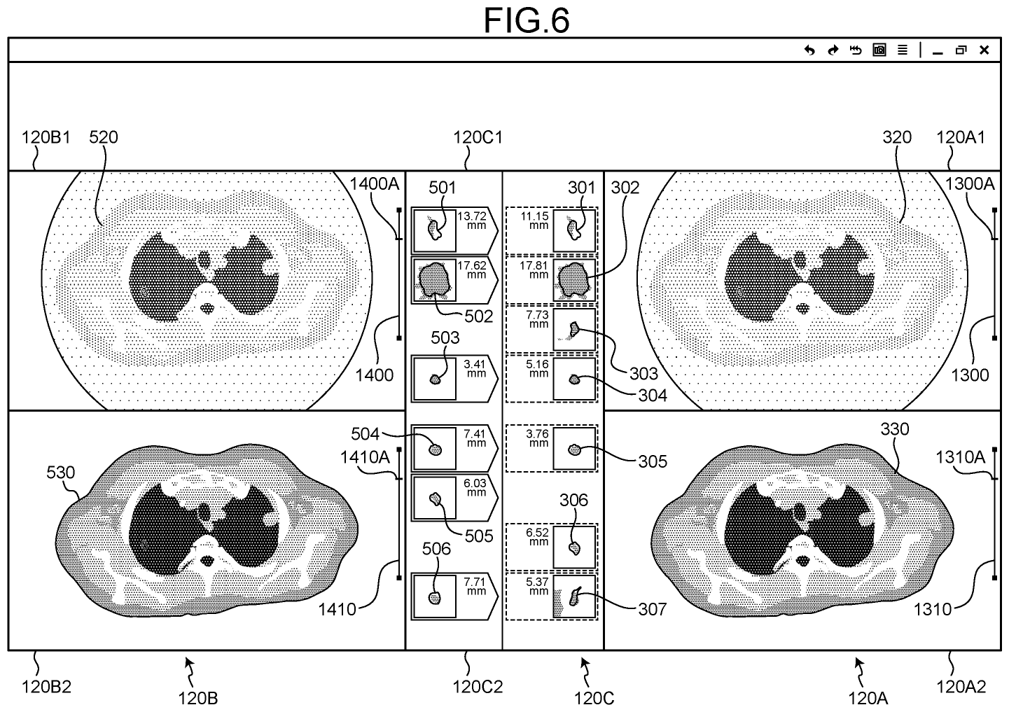

A process performed by the medical image processing apparatus 100 according to the second embodiment is described below with reference to FIGS. 3 and 6. FIG. 6 is a diagram illustrating a display example of the medical image processing apparatus 100 according to the second embodiment.

At step S101 in FIG. 3, the acquisition function 151 acquires the current image and the image n times before, which are medical images with different imaging times, with respect to a target site of the subject. For example, suppose that a user has given an instruction to acquire the current image and the image n times before by using the input interface 110. In this case, the acquisition function 151 acquires, from the image storage apparatus 3, along with the current image and the image n times before, the patient information including a patient ID, a patient name, and the like for identifying the subject (patient), the date the current image and the image n times before have been taken, and the like.

The current image is an example of the “first image” and the image n times before is an example of the “second image”. Also in the second embodiment, suppose that the target site of the subject is the lungs.

At step S102 in FIG. 3, the setting function 152 sets a plurality of regions of interest in the current image and the image n times before.

At step S103 in FIG. 3, the specifying function 153 specifies pairs of corresponding regions of interest in the current image and the image n times before among a plurality of regions of interest 301 to 307 and 501 to 506.

For example, by using the anatomical location information of an images of the lung field condition being the current image and the image n times before, the specifying function 153 specifies the region of interest 301 in the current image and the region of interest 501 in the image n times before as a pair and specifies the region of interest 302 in the current image and the region of interest 502 in the image n times before as a pair. For example, by using the anatomical location information of the images of the lung field condition being the current image and the image n times before, the specifying function 153 specifies the region of interest 304 in the current image and the region of interest 503 in the image n times before as a pair.

For example, by using the anatomical location information of an images of the lung field condition being the current image and the image n times before, the specifying function 153 specifies the region of interest 305 in the current image and the region of interest 504 in the image n times before as a pair and specifies the region of interest 307 in the current image and the region of interest 506 in the image n times before as a pair.

The region of interest 303 in the image of the lung field condition, which is the current image, is a region of interest for which no pair has been specified. For example, the region of interest 303 is a newly set region of interest in the current image. The region of interest 306 in the image of the lung field condition, which is the current image, and the region of interest 505 in the image of the lung field condition, which is the image n times before, are regions of interest for which no pair has been specified. For example, the region of interest 306 is a newly set region of interest in the current image, and the region of interest 306 in the current image does not correspond to the region of interest 505 in the image n times before.

At step S104 in FIG. 3, the display control function 154 causes the display 120 to display the current image and the image n times before, and also causes the display 120 to display information corresponding to each of the plurality of regions of interest in the current image and the image n times before in a manner that distinguishes the regions of interest for which the above pair has been specified from the regions of interest for which the above pair has not been specified.

For example, the display control function 154 displays an image 320 of the lung field condition and an image 330 of the mediastinal condition, which are the current images, in the display area 120A. The display control function 154 further displays an image 520 of the lung field condition and an image 530 of the mediastinal condition, which are the images n times before, in the display area 120B.

In the example illustrated in FIG. 6, the display control function 154 displays information corresponding to each of the plurality of regions of interest 301 to 307 and 501 to 506 in the display area 120C in a manner that the regions of interest 301 and 501, the regions of interest 302 and 502, the regions of interest 304 and 503, the regions of interest 305 and 504, and the regions of interest 307 and 506, for which the above pair has been specified, are distinguished from the regions of interest 303, 306, and 505 for which the above pair has not been specified. The above information includes, for example, at least the diameter of the region of interest (nodule) and an image of the region of interest.

Specifically, in FIG. 6, the display control function 154 arranges and displays, in the display area 120C, pairs of information (diameters and images of nodules) corresponding to the regions of interest 301 and 501, the regions of interest 302 and 502, the regions of interest 304 and 503, the regions of interest 305 and 504, and the regions of interest 307 and 506, for which the above pair has been specified.

For example, the display control function 154 arranges and displays, in the upper stage 120C1 of the display area 120C, the pairs of the information (diameters and images of nodules) corresponding to the region of interest 301 in the current image and the region of interest 501 in the image n times before as the region of interest for which the above pair has been specified in an I^{th} slice image (I is an integer of 2 or more) being the image of the lung field condition. The display control function 154 further arranges and displays, in the upper stage 120C1 of the display area 120C, the pairs of the information (diameters and images of nodules) corresponding to the region of interest 302 in the current image and the region of interest 502 in the image n times before as the region of interest for which the above pair has been specified in a J^{th} slice image (J is an integer satisfying I<J) being the image of the lung field condition. The display control function 154 further arranges and displays, in the upper stage 120C1 of the display area 120C, the pairs of the information (diameters and images of nodules) corresponding to the region of interest 304 in the current image and the region of interest 503 in the image n times before as the region of interest for which the above pair has been specified in an M^{th} slice image (M is an integer satisfying J<M) being the image of the lung field condition. The display control function 154 displays the region of interest 303 in the current image in the upper stage 120C1 of the display area 120C as the region of interest for which the above pair has not been specified in a K^{th} slice image (K is an integer satisfying J<K<M) being the image of the lung field condition.

The display control function 154 further arranges and displays, in the lower stage 120C2 of the display area 120C, the pairs of the information (diameters and images of nodules) corresponding to the region of interest 305 in the current image and the region of interest 504 in the image n times before as the region of interest for which the above pair has been specified in an Nth slice image (N is an integer of 2 or more) being the image of the lung field condition. The display control function 154 further arranges and displays, in the lower stage 120C2 of the display area 120C, the pairs of the information (diameters and images of nodules) corresponding to the region of interest 307 in the current image and the region of interest 506 in the image n times before as the region of interest for which the above pair has been specified in an R^{th} slice image (R is an integer satisfying N<R) being the image of the lung field condition. The display control function 154 displays the region of interest 505 in the image n times before in the lower stage 120C2 of the display area 120C as the region of interest for which the above pair has not been specified in a P^{th} slice image (P is an integer satisfying N<P<R) being the image of the lung field condition. The display control function 154 further displays the region of interest 306 in the current image in the lower stage 120C2 of the display area 120C as the region of interest for which the above pair has not been specified in a Q^{th} slice image (Q is an integer satisfying P<Q<R) being the image of the lung field condition.

Thus, in the image of the lung field condition, the user can confirm whether the diameter of the region of interest in the current image is decreasing or increasing compared to the diameter of the region of interest in the image n times before, or whether the difference between the diameters is small. In the image of the lung field condition, the user can newly confirm a region of interest in the current image, and confirm the diameter of the region of interest.

In this way, in the medical image processing apparatus 100 according to the present embodiment, as in the first embodiment, the display 120 displays the information (diameters and images of nodules) corresponding to each of the plurality of regions of interest, so that the user can easily compare the sizes of the regions of interest in the image n times before and the current image when performing comparative reading. Therefore, in accordance with the medical image processing apparatus 100 according to the present embodiment, the efficiency of reading can be improved.

### Third Embodiment

In the third embodiment, the same explanations as in the second embodiment are omitted.

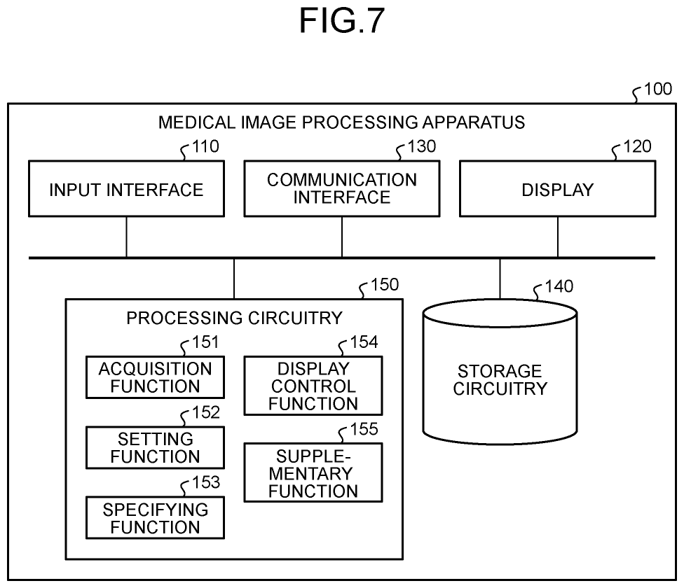

FIG. 7 is a diagram illustrating an example of the configuration of a medical image processing apparatus 100 according to the third embodiment. As illustrated in FIG. 7, the processing circuitry 150 of the medical image processing apparatus 100 performs the acquisition function 151, the setting function 152, the specifying function 153, the display control function 154, and a supplementary function 155. That is, the processing circuitry 150 further performs the supplementary function 155. The supplementary function 155 is an example of a “supplementary unit.

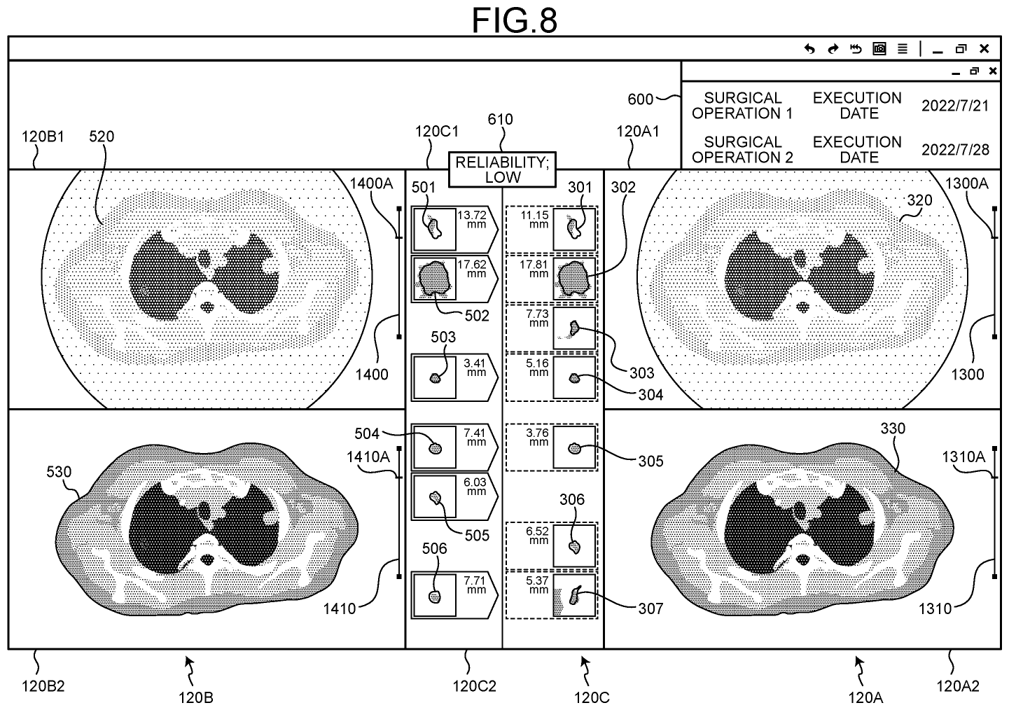

A process performed by the medical image processing apparatus 100 according to the third embodiment is described below with reference to FIGS. 3 and 8. FIG. 8 is a diagram illustrating a display example of the medical image processing apparatus 100 according to the third embodiment. The third embodiment differs from the second embodiment in that supplementary information is provided.

A supplementary information display process is described below with reference to FIGS. 3 and 8. Since N is an integer equal to or greater than 1 in the third embodiment as well, for example, when N is set to 1, the supplementary information display process can be implemented in the comparative reading between the current image and the previous image (image one time before) described in the first embodiment.

First, the supplementary function 155 supplements, as supplementary information, clinical information of a subject at the time phase, in which an image has been acquired, to at least one of the current image (image of the lung field condition and image of the mediastinal condition) and the image n times before (image of the lung field condition and image of the mediastinal condition). The specifying function 153 determines the reliability of specified pairs of regions of interest based on the supplementary information, and the display control function 154 causes the display 120 to display reliability-related information along with the information (diameters and images of nodules) corresponding to each of the plurality of regions of interest.

First, at step S101 in FIG. 3, the acquisition function 151 acquires the current image and the image n times before, which are medical images with different imaging times, with respect to a target site of the subject. For example, suppose that a user has given an instruction to acquire the current image and the image n times before by using the input interface 110. In this case, the acquisition function 151 acquires, from the image storage apparatus 3, along with the current image and the image n times before, the patient information including a patient ID, a patient name, and the like for identifying the subject (patient), the date “Sep. 3, 2022” the current image has been taken, the date “May 28, 2022” the image n times before has been taken, and the like.

The current image is an example of the “first image” and the image n times before is an example of the “second image”. Also in the third embodiment, suppose that the target site of the subject is the lungs.

At step S101, the supplementary function 155 acquires the clinical information of the subject at the time phase, in which the image has been acquired, as supplementary information 600 (see FIG. 6) with respect to the current image and the image n times before. For example, the supplementary function 155 acquires a medical event, which has occurred on “Jul. 21, 2022” between the date “May 28, 2022” the image n times before has been taken and the date “Sep. 3, 2022” the current image has been taken, from the HIS server 10 as the supplementary information 600.

Subsequently, at step S102 in FIG. 3, the setting function 152 sets a plurality of regions of interest in the current image and the image n times before.

Subsequently, at step S103 in FIG. 3, the specifying function 153 specifies pairs of corresponding regions of interest in the current image and the image n times before among the plurality of regions of interest 301 to 307 and 501 to 506. In the example illustrated in FIG. 8, the specifying function 153 specifies pairs of the regions of interest 301 and 501, the regions of interest 302 and 502, the regions of interest 304 and 503, the regions of interest 305 and 504, and the regions of interest 307 and 506 in the current image and the image n times before.

At step S103, the specifying function 153 confirms the supplementary information 600 acquired by the supplementary function 155. Specifically, on the basis of the supplementary information 600 acquired by the supplementary function 155, the specifying function 153 confirms whether a medical event affecting the reliability of the specified pairs of the regions of interest has been performed.

For example, the clinical information of the subject at the time phase in which the image has been acquired is a medical event significantly affecting the body of the subject. For example, the medical event is a treatment significantly affecting the shape of the body, such as a surgical operation performed on the subject. In this case, the treatment causes a change in the shape of the body of the subject.

By the execution of the medical event, the specifying function 153 determines the reliability of the specified pair of regions of interest. In the example illustrated in FIG. 8, the specifying function 153 determines the reliability of the pairs of the regions of interest 301 and 501, the regions of interest 302 and 502, the regions of interest 304 and 503, the regions of interest 305 and 504, and the regions of interest 307 and 506 in the current image and the image n times before.

For example, the specifying function 153 determines the reliability of the specified pairs of the regions of interest from the supplementary information 600 by referring to a table that associates medical events with the reliability of the specified pairs of the regions of interest. For example, the medical events are listed in the table and stored in storage circuitry 140. When the medical event is a treatment that causes a change in the shape of the body of the subject, the table is set to have a lower reliability determination result for the pair of regions of interest specified by the specifying function 153. On the other hand, when the medical event is a treatment that is unlikely to cause a change in the shape of the body of the subject, the table is set to have a higher reliability determination result for the pair of regions of interest specified by the specifying function 153.

For example, time phases in which images have been acquired are the date “Sep. 3, 2022” the current image has been taken and the date “May 28, 2022” the image n times before has been taken. The following description will be given on the assumption that for example, on “Jul. 21, 2022” and “Jul. 28, 2022” between the date “May 28, 2022” the image n times before has been taken and the date “Sep. 3, 2022” the current image has been taken, surgical operations “Surgical operation 1” and “Surgical operation 2” have occurred in the subject as medical events, respectively. For example, suppose that information on the “Surgical operation 1” performed on the subject on “Jul. 21, 2022” and information on the “Surgical operation 2” performed on the subject on “Jul. 28, 2022” are recorded in an electronic medical record managed by the HIS server 10, as treatments significantly affecting the shape of the body.

In this case, the specifying function 153 determines that the medical events of the surgical operations occurred on “Jul. 21, 2022” and “Jul. 28, 2022” as medical events are medical events affecting reliability, determines the reliability of the specified pair of regions of interest to be low, and requests the display control function 154 to display, on the display 120, the supplementary information 600 and reliability-related information 610 “Reliability; Low”.

Subsequently, at step S104 in FIG. 3, the display control function 154 causes the display 120 to display the current image and the image n times before, and causes the display 120 to display information corresponding to each of the plurality of regions of interest between the current image and the image n times before in a manner that distinguishes the regions of interest for which the above pair has been specified from the regions of interest for which the above pair has not been specified. In the example illustrated in FIG. 8, the display control function 154 causes the display 120 to display, as the supplementary information 600, the fact that the execution dates “Jul. 21, 2022” and “Jul. 28, 2022”, on which the surgical operations “Surgical operation 1” and “Surgical operation 2” have been performed on the subject, respectively, are located between the date “May 28, 2022” the image n times before has been taken and the date “Sep. 3, 2022” the current image has been taken. In the example illustrated in FIG. 8, the display control function 154 displays the reliability-related information 610 “Reliability; Low” in the display area 120C of the display 120, along with the information (diameters and images of nodules) corresponding to each of the plurality of regions of interest 301 to 307 and 501 to 506.

In the example illustrated in FIG. 8, the display control function 154 causes the display 120 to display the supplementary information 600 and the reliability-related information 610 “Reliability; Low”, but the display control function 154 may cause the display 120 to display only a medical event affecting the reliability of the specified pair of regions of interest.

The clinical information of the subject is not limited to the medical event described above, but is also applied, for example, when a significant change exists in physical information (weight and the like) of the subject. In this case, the supplementary function 155 acquires, from the HIS server 10 as the supplementary information 600, that a significant change exists in the physical information (weight and the like) of the subject in the information recorded in the electronic medical record on “Jul. 21, 2022” between the date “May 28, 2022” the image n times before has been taken and the date “Sep. 3, 2022” the current image has been taken. On the basis of the supplementary information 600, the specifying function 153 determines the reliability of the specified pair of regions of interest, and on the basis of the result of the determination by the specifying function 153, the display control function 154 displays reliability-related information in the display area 120C of the display 120.

The specifying of the pairs of the regions of interest by the specifying function 153 may be performed when the user (operator) designates pairs of the regions of interest in the current image (image of the lung field condition and image of the mediastinal condition) and the image n times before (image of the lung field condition and image of the mediastinal condition) displayed on the display 120.

For example, the specifying function 153 refers to the region of interest 306 and the region of interest 505 as regions of interest for which no pair has not been specified in the current image and the image n times before; however, actually, the region of interest 306 and the region of interest 505 may be a pair of corresponding regions of interest in the current image and the image n times before.

In this case, when the display control function 154 is displaying the current image and the previous image on the display 120, the user operates the input interface 110 to designate a pair of the region of interest 306 in the current image and the region of interest 505 in the image n times before. Specifically, the user operates the input interface 110 to select information (diameters and images of nodules) corresponding to the region of interest 306 in the display area 120C, and to arrange the selected information with information (diameters and images of nodules) corresponding to the region of interest 505. In this case, the specifying function 153 specifies a pair of the region of interest 306 and the region of interest 505 in the current image and the image n times before, and the display control function 154 arranges and displays, in the lower stage 120C2 of the display area 120C, the pairs of the information (diameters and images of nodules) corresponding to the region of interest 306 in the current image and the region of interest 505 in the image n times before.

In this way, the medical image processing apparatus 100 according to the present embodiment associates clinical information (supplementary information 600) of a subject at the time phase in which an image has been acquired, and causes the display 120 to display information (diameters and images of nodules) corresponding to each of a plurality of regions of interest, along with the reliability of specified pairs of regions of interest. Thus, the user can perform comparative reading while taking into account the clinical information of the subject. Therefore, the medical image processing apparatus 100 according to the present embodiment can assist in the observation of medical images.

In the present embodiment, in FIG. 8, the regions of interest 303 and 306 in the image of the lung field condition that is the current image and the region of interest 505 in the image of the lung field condition that is the image n times before are regions of interest for which no pair has been specified, but are not limited to these regions. For example, the display control function 154 may cause information (diameters and images of nodules) corresponding to a region of interest for which no pair has been specified in one of the current image and the image n times before to be displayed near information (diameters and images of nodules) corresponding to a region of interest that is anatomically close to a region of interest in the other of the current image and the image n times before.

For example, in the present embodiment, in FIG. 8, the region of interest 307 in the current image and the region of interest 506 in the image n times before are specified as a pair. When the region of interest 306 in the current image is also anatomically close to the region of interest 506 in the image n times before, the display control function 154 causes information (diameters and images of nodules) corresponding to the region of interest 506 to be displayed near information (diameters and images of nodules) corresponding to the region of interest 307. Thus, in the present embodiment, the user is made to recognize the pair of the region of interest 307 and the region of interest 506 in the current image and the image n times before, and to notice that the region of interest 306 is also anatomically close to the region of interest 506. That is, the present embodiment can give the user an opportunity to modify the pair of regions of interest by making the user aware that the region of interest 306 is also anatomically close to the region of interest 506.

For example, the region of interest 306 in the current image and the region of interest 506 in the image n times before could not have been specified as a pair due to factors such as a misalignment in a body axis direction or a misalignment in an axial section. Therefore, as a result of image alignment using anatomical location information, the specifying function 153 specifies a region of interest (for example, the region of interest 506), which is anatomically close to a non-specified region of interest being a region of interest (for example, the region of interest 306) for which no pair has not been specified in one (for example, the current image) of the current image and the image n times before, in the other (for example, the image n times before) of the current image and the image n times before.

In this case, the display control function 154 causes the display 120 to display the current image and the image n times before, and also causes the display 120 to display information corresponding to each of a plurality of regions of interest between the current image and the image n times before in a manner that distinguishes regions of interest for which a pair has been specified from regions of interest for which no pair has been specified. At this time, the display control function 154 causes information (diameters and images of nodules) corresponding to a non-specified region of interest (region of interest 306) in the above one image (current image) to be displayed near the information (diameters and images of nodules) corresponding to the region of interest 506 specified in the above other image (image n times before), in the lower stage 120C2 of the display area 120C of the display 120. Thus, the present embodiment allows the user aware that the region of interest 306 in the current image is also anatomically close to the region of interest 506 in the image n times before, and also encourages the user to determine whether to modify the pair of regions of interest to the pair of the region of interest 306 in the current image and the region of interest 506 in the image n times before.

For example, suppose that the user determines to modify the pair of regions of interest to the pair of the region of interest 306 in the current image and the region of interest 506 in the image n times before. In this case, the user can pair regions of interest by using the input interface 110. For example, the user operates the input interface 110 to designate the pair of the region of interest 306 in the current image and the region of interest 506 in the image n times before. For example, the user operates the input interface 110 to select the information (diameters and images of nodules) corresponding to the region of interest 306 in the display area 120C, and to arrange the selected information with the information (diameters and images of nodules) corresponding to the region of interest 506. In this case, the specifying function 153 specifies the pair of the region of interest 306 and the region of interest 506 in the current image and the image n times before by the user operation, and the display control function 154 arranges and displays, in the lower stage 120C2 of the display area 120C, the pairs of the information (diameters and images of nodules) corresponding to the region of interest 306 in the current image and the region of interest 506 in the image n times before.

Each component of each apparatus illustrated in the present embodiment is functionally conceptual, and does not necessarily have to be physically configured as illustrated in the drawings. That is, the specific form of distribution or integration of each apparatus is not limited to those illustrated in the drawings, but can be configured by functionally or physically distributing or integrating all or part thereof in arbitrary units, depending on various loads, use conditions, and the like. Moreover, each processing function performed by each apparatus can be implemented in whole or in part by a CPU and a computer program that is analyzed and executed by the CPU, or by hardware using wired logic.

The method described in the present embodiment can be implemented by executing a computer program prepared in advance on a computer such as a personal computer or a workstation. The computer program can be distributed via a network such as the Internet. The computer program can also be recorded on a non-transitory computer readable recording medium such as a hard disk, a flexible disk (FD), a CD-ROM, an MO, and a DVD, and can be executed by being read from the recording medium by a computer.

According to at least one embodiment described above, the efficiency of reading can be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured to
acquire a first image and a second image with respect to a target site of a subject, the first image and the second image being medical images with different imaging times,
set a plurality of regions of interest in the first image and the second image,
specify pairs of corresponding regions of interest in the first image and the second image among the plurality of regions of interest, and
cause a display to display the first image and the second image, and cause the display to display information corresponding to each of the plurality of regions of interest between the first image and the second image in a manner such that regions of interest for which the pair has been specified are distinguished from regions of interest for which the pair has not been specified.

2. The medical image processing apparatus according to claim 1, wherein the processing circuitry causes the display to arrange and display pairs of the information corresponding to the regions of interest for which the pair has been specified.

3. The medical image processing apparatus according to claim 1, wherein the processing circuitry causes the display to display at least an image of the region of interest as the information corresponding to each of the plurality of regions of interest.

4. The medical image processing apparatus according to claim 1, wherein
the setting of the region of interest by the processing circuitry is performed when an operator designates an area in the first image or the second image displayed on the display, and
the processing circuitry causes the display to display an enlarged image corresponding to the area in response to the designation of the area.

5. The medical image processing apparatus according to claim 1, wherein
the processing circuitry receives selection of the information made by an operator,
at least one of the first image and the second image is a three-dimensional medical image obtained by imaging a three-dimensional area of the subject, and
when a region of interest corresponding to the information for which the selection has been received is a region set by the processing circuitry in the three-dimensional medical image, the processing circuitry causes the display to display a cross-section including the region of interest in the three-dimensional medical image.

6. The medical image processing apparatus according to claim 1, wherein the processing circuitry
supplements, as supplementary information, clinical information of the subject at a time phase, in which an image has been acquired, to at least one of the first image or the second image,
determines a reliability of the pairs of the specified regions of interest based on the supplementary information, and
causes the display to display information on the reliability along with the information.

7. The medical image processing apparatus according to claim 1, wherein the processing circuitry
specifies a region of interest anatomically close to a non-specified region of interest being a region of interest, for which no pair has not been specified in one of the first image or the second image, in the other of the first image or the second image, and
causes the information corresponding to the non-specified region of interest to be displayed near the information corresponding to a region of interest specified in the other image.

8. A non-transitory computer readable medium comprising instructions that cause a computer to execute:
acquiring a first image and a second image with respect to a target site of a subject, the first image and the second image being medical images with different imaging times,
setting a plurality of regions of interest in the first image and the second image,
specifying pairs of corresponding regions of interest in the first image and the second image among the plurality of regions of interest, and
causing a display to display the first image and the second image, and causing the display to display information corresponding to each of the plurality of regions of interest between the first image and the second image in a manner such that regions of interest for which the pair has been specified are distinguished from regions of interest for which the pair has not been specified.
